(19) United States
(12) Patent Application Publication
(10) Pub. No.: US 2013/0018591 A1
(43) Pub. Date: Jan. 17, 2013

(54) FAST TOMOGRAPHIC MICROWAVE IMAGING

(71) Applicant: Tomasz M. Grzegorczyk, Newton, MA (US)

(72) Inventor: Tomasz M. Grzegorczyk, Newton, MA (US)

(21) Appl. No.: 13/546,191

(22) Filed: Jul. 11, 2012

Related U.S. Application Data
(60) Provisional application No. 61/508,685, filed on Jul. 17, 2011.
(60) Provisional application No. 61/515,854, filed on Aug. 6, 2011.

(51) Int. Cl. G06F 19/00; G06K 9/00; G01N 33/48

(52) U.S. Cl. 702/19; 382/131

(57) ABSTRACT

Microwave imaging equipment utilizing an array of antennas operated to collect electromagnetic field information for a material being imaged. Image processing method and apparatus use the discrete dipole approximation (DDA) and drastically reduce the time required to process the measured data and estimate the properties of the material. Prior to interrogating the material, interaction matrices are generated and stored for future DDA calculations. The interaction matrices relate to the interaction between the antennas, the operating frequency, the background medium, and the location of the discretizing dipoles. An initial guess of the material properties is made and the resultant field is estimated. These results are compared to the measured results and incremental changes in the material property are computed. The updated material properties are used to recalculate the field. Comparison of the field to the measured field and updating of the material properties continues until an end criterion is satisfied.

The present application claims priority under 35 U.S.C. §119(e) to U.S. provisional patent applications, U.S. Ser. No. 61/508,685 filed on Jul. 17, 2011, and 61/515,854 filed on Aug. 6, 2011, each of which is herein incorporated by reference in its entirety.

### BACKGROUND

Biomedical imaging is experiencing an increased interest among research institutions, pharmaceutical companies, and hospitals due to unique properties such as low cost, non-invasiveness, wide applicability range, and potential good sensitivity and specificity. Most clinical imaging modalities are currently based on the interaction of either electromagnetic or acoustic waves with body tissues and fluids. In the realm of electromagnetic waves, a large portion of the spectrum has already been exploited, from very high frequencies (positron emission tomography, X-ray/computed tomography), to lower and non-ionizing frequencies such as infrared, near-infrared, THz, and low MHz regions where magnetic resonance (MR) operates. A portion of the spectrum much less investigated is that of microwave frequencies, from a few hundreds of MHz to several GHz. Electromagnetic waves, the means by which microwaves transport power and information, are well understood and their propagation can be accurately simulated in a large variety of environments. The interaction of electromagnetic waves and matter depends on dielectric properties which can be directly related to various types of biological constituents due to their varying degree of water content: bone, fat, muscle, etc. This specificity can be exploited in different tissue types and even offers the rationale for detecting tumors: ex vivo studies have revealed varying degrees of contrast in the dielectric properties between normal and malignant tissues. The permittivity is normally high at lower frequencies due to the insulating effect of cell membranes, and decreases over higher frequencies due to dispersion and relaxation. Low water content tissues include for example fat, bone, and lung, whereas high water content tissues include muscles, brain, blood, internal organs, and tumors. For low water-content tissues such as the breast, studies indicate that tumor versus normal tissues property contrast can range from 10% to 400%, sufficiently large to be captured by microwave imaging.

Among various possible applications, breast cancer imaging remains a high research priority because of its vast incidence. Roughly 200,000 new cases of breast cancer are typically diagnosed in the U.S. every year, with an estimated 25% to 30% of women dying from the disease making it the second largest cause of female cancer deaths in the U.S. There have been numerous reports demonstrating that early detection is the single most significant predictor of long term survival; therefore, improvements in detection may help reduce the current high mortality rates. Mammography is the front line screening modality but its weaknesses in terms of sensitivity and specificity are well documented. While magnetic resonance and ultrasound are currently used primarily in a diagnostic setting, cost issues for the former and resolution for the latter prevent these techniques from being accessible to a broad population. Notwithstanding, the morphology of breast tissue is ideal for microwave imaging: fibroglandular tissue has been shown to have higher dielectric properties than fat but with properties that are still lower than tumors in most cases. Furthermore, studies have shown that the amount of fibroglandular tissue can vary widely from fattier breasts (very little) to denser breasts (much larger proportions with highly heterogeneous tissue mixture) suggesting that the baseline electrical properties of the normal breast may be highly variable.

The microwave imaging techniques presently being pursued are generally either radar or tomography. With respect to radar technology, the most advanced concepts appear to be confocal microwave imaging and near field synthetic focusing. Most studies of microwave tomography are occurring at the simulation and prototype stages where the goal is to identify and optimize the design rather than report on electrical characteristics of breast tissues per se. In addition, most of the microwave breast cancer detection algorithms currently used in conjunction with actual hardware are burdened with significant computational times which currently limit their clinical utility. Despite nearly two decades of research and development, it is not uncommon to wait tens of hours or even days for a single 3D microwave tomographic image.

### SUMMARY

Microwave imaging equipment utilizes an array of antennas which are operated in various configurations of transmitter and receivers to collect field information that may be converted into the material properties of the area being imaged. In many potential application settings, such as for a clinical breast examination, civil engineering applications, and others, it is important that the interrogation is quickly performed and the results quickly processed. Image processing is described that drastically reduces the time required to process the measured data and estimate the properties of the interrogated material. In an example application of female breast imaging for the detection of tumors, the imaging time was reduced to under 10 minutes, as compared to prior systems which took several hours to provide imaging results.

The image processing approach makes use of the discrete dipole approximation (DDA). Prior to interrogating the test material, interaction matrices are generated and stored for use in future DDA calculations. The interaction matrices relate to the interaction between the transmitters, receivers, and the dipole locations defined for the DDA. An initial guess of the material properties in the imaging region is made and the resultant field is estimated using DDA. These results are compared to the measured results and incremental changes in the material property are computed. In determining the material property updates, the Jacobian matrix may be computed analytically, numerically, semi-analytically or in any other suitable way. In some embodiments the Gauss-Newton method is utilized. The updated material properties are used to recalculate the field. Comparison of the field to the measured field and updating of the material properties is continued until a suitable criteria is met, at which time the material properties may be presented or post processed to determine the presence of a feature of interest.

Some aspects relate to a non-transitory, computer-readable storage medium having computer-executable instructions that, when executed perform a method of imaging a test material from microwave measurement data, the method comprising acts of (a) receiving the microwave measurement data; (b) computing a field at each of a plurality of dipole locations and at each of a plurality of receiver locations for a guess of material properties; (c) computing a Jacobian matrix using the computed field and the guess of the material properties; (d) estimating a total phase difference at each of the plurality of receiver locations from the computed scattered field and a phase calculated for a homogeneous medium; (e)determining a new guess of the material properties based on the total phase difference, the Jacobian matrix and the received measurement data; (f) repeating steps (b) through (e) until an end criteria is met; and (g) outputting a representation of the material properties.

In some embodiments of the computer-readable storage medium the measurement data comprises measured electromagnetic fields at each of the plurality of receiver locations. The measured electromagnetic fields may have a frequency between 50 MHz and 20 GHz.

In some embodiments of the computer-readable storage medium, the act (c) comprises computing the Jacobian matrix analytically.

In some embodiments of the computer-readable storage medium the act (b) comprises loading one or more precomputed interaction matrices and computing the scattered field from the guess of the material properties and the one or more precomputed interaction matrices. The one or more precomputed interaction matrices may comprise an interaction matrix between all dipoles in order to compute the scattered field, an interaction matrix between the transmitters and receivers in order to compute the incident field at each receiver, and an interaction matrix between the transmitters and all the dipoles, in order to compute the incident field from a transmitter at a given dipole location.

In some embodiments of the computer-readable storage medium the act (b) comprises positioning each of the plurality of dipoles on a regular grid and using a fast Fourier transform to expedite matrix multiplication.

In some embodiments of the computer-readable storage medium the act (b) comprises positioning each of the plurality of dipoles on a regular grid and simplifying execution by exploitation of a matrix Block-Toeplitz format.

In some embodiments of the computer-readable storage medium the method is for imaging a test material that is a human, female breast.

In some embodiments of the computer-readable storage medium the method is for imaging a test material that is a civil engineering structure.

In some embodiments of the computer-readable storage medium in the act (f) the end condition is a convergence criteria for the computed fields and measurement data.

In some embodiments of the computer-readable storage medium the method of imaging the test material further comprises receiving position information for the test material volume; identifying dipoles positioned outside the test material volume; and fixing the material properties of each of the dipoles identified as outside the test material volume to the material properties of a background material.

Another aspect relates to a non-transitory, computer-readable storage medium having computer-executable instructions that, when executed perform a method of imaging material properties of a test material from microwave measurement data. The method comprising acts of (a) receiving the microwave measurement data; (b) translating the microwave measurement data into material properties of the test material by: (i) estimating fields for a guess of the material properties using a discrete dipole approximation (DDA), (ii) updating the guess by comparing the estimated fields with the microwave measurement data, (iii) iterating (i) and (ii) until an end condition is met; and (c) outputting a representation of the material properties.

In some embodiments of the computer-readable storage medium the guess is updated at (b)(ii) using a non-linear solver. For example, the guess may be updated at (b)(ii) using a Gauss-Newton method. In some embodiments, a Jacobian matrix is evaluated analytically for the Gauss Newton method.

In some embodiments of the computer-readable storage medium the material properties imaged by the method comprise at least one of permittivity and conductivity of the test material.

In some embodiments of the computer-readable storage medium at (b)(ii) the end condition is a convergence criteria for the estimated fields and measured data.

In some embodiments of the computer-readable storage medium outputting a representation of the material properties comprises providing an image of the material properties in a human viewable format.

In some embodiments of the computer-readable storage medium translating the microwave measurement data into material properties comprises separately imaging each of a plurality of image planes using separately computed dipoles; and forming a 3D representation of the image region by combing each of the separately imaged planes using a weighted average of the dipole material properties computed for each separate image. For example, each image plane may be obtained from measurement data obtained from different antenna heights. As another example, the plurality of dipoles for each separately imaged plane may cover a region smaller than the overall 3D imaging region.

In some embodiments of the computer-readable storage medium translating the microwave measurement data into material properties comprises generating a 3D representation of the material properties.

In some embodiments of the computer-readable storage medium translating the microwave measurement data into material properties further comprises forming a 2D image by directionally averaging in a direction of interest; and providing an image of the material properties in a human viewable format. For example, the direction of interest may be one of a coronal, sagittal, or axial directions.

Yet another aspect relates to a method of constructing a plurality of interaction matrices. The method comprises operating a processor to define a plurality of dipole locations; compute a plurality of interaction matrices for the dipole locations; and store the plurality of interaction matrices on a non-transitory computer readable storage medium.

In some embodiments of the method, operating the processor to compute the plurality of interaction matrices comprises computing a first interaction matrix between all dipoles in order to compute the scattered field; computing a second interaction matrix between the transmitters and receivers in order to compute the incident field at each receiver; and computing a second interaction matrix between the transmitters and all the dipoles, in order to compute the incident field from a transmitter at a given dipole location.

In some embodiments of the method the plurality of interaction matrices are computed for a plurality of different excitation frequencies.

In some embodiments of the method the plurality of interaction matrices are computed for each of a plurality of different dipole densities.

In some embodiments of the method the plurality of interaction matrices are computed for each of a plurality of different antenna configurations.

In some embodiments of the method, the plurality of interaction matrices are computed for each of a plurality of different background media.

In some embodiments of the method the dipole positions are on a regular grid. In some embodiments the dipole positions are on a cubic lattice. In some embodiments of the method the dipole positions are irregular.

Another aspect relates to a method of operating a computer. The method comprises operating a processor to (a) receive microwave measurement data; (b) translate the microwave measurement data into material properties of a test material by (i) estimating fields for a guess of the material properties using a discrete dipole approximation (DDA), (ii) updating the guess by comparing the estimated fields with the microwave measurement data, (iii) iterating (i) and (ii) until an end condition is met; and (c) outputting a representation of the material properties.

Another aspect relates to an image processing device comprising a processor configured to perform acts of (a) receiving the microwave measurement data; (b) translating the microwave measurement data into material properties of the test material by (i) estimating fields for a guess of the material properties using a discrete dipole approximation (DDA), (ii) updating the guess by comparing the estimated fields with the microwave measurement data, (iii) iterating (i) and (ii) until an end condition is met; and (c) outputting a representation of the material properties.

It should be appreciated that the foregoing is intended to be a non-limiting summary of the invention, which is defined only by the attached claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a block diagram of an imaging system, according to some embodiments;

FIG. 2 is a flow diagram of a method for precomputing interaction matrices, according to some embodiments;

FIG. 3 is an illustration of a dipole configuration, according to some embodiments; and

FIG. 4 is a flow diagram of a method for generating an image from measurement data.

### DETAILED DESCRIPTION

The inventor has recognized and appreciated that conventional microwave imaging systems have been limited in application, in part due to the extensive processing time required for image reconstruction. As discussed above, prior art systems can require tens of hours to reconstruct images. In many practical applications of microwave imaging the long processing time may not be practical. For example, one factor that prevented the use of the modality for a clinical breast examination is the processing delay. A tomographic microwave image processing device is described in the context of imaging of a human female breast for the purposes of detection of tumors. It should be appreciated that this application is merely exemplary and the invention is not so limited. Other applications include bone imaging, thermal imaging, brain activation imaging, other anatomical parts (human or otherwise), other biomedical imaging applications, timber imaging, imaging of levees and dams, security imaging, civil engineering imaging, and imaging of composite media and materials. These are examples and are non-limiting as the described image processing device and methods may be used for any suitable application.

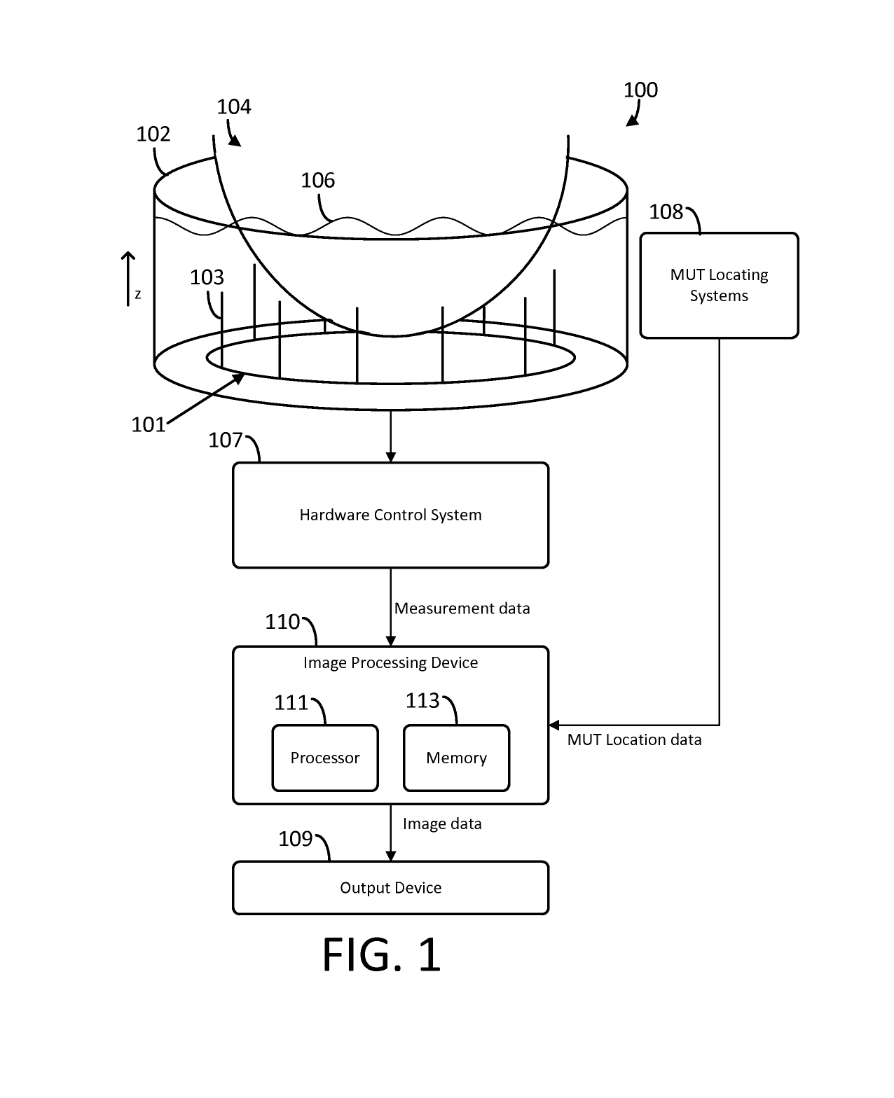

FIG. 1 shows an imaging system 100 with the disclosed image processing device 110. Imaging system 100 provides two-dimensional (2D) or three-dimensional (3D) imaging of a breast in minutes on commercially available computers. As compared with prior art systems, this is a drastic decrease in processing time which is expected to make microwave imaging a more practical solution for clinical breast imaging and other microwave imaging applications. Image processing device 110 may replace existing image processing solutions in conventional tomographic microwave imaging systems such as that described in U.S. Pat. No. 5,841,288 to Meaney et al. (hereinafter Meaney '288). (Full Citations to all references are provided at the end of the Detailed Description.)

Imaging system 100 has an imaging array 101 for interrogating a material under test (MUT) 104. In the described embodiment the MUT is a human female breast. Though the MUT may be, for example and not limitation, bone, brain matter, other anatomical parts (human or otherwise), timber, a levee, dam or other civil engineering structure, composite material, or any other suitable test material. Measurements may be from a phantom, a biological tissue, or any other entity to be imaged. Imaging array 101 has a plurality of antenna elements 103 that may be arranged on a regular grid, define a regular or irregular shape, have irregular locations (e.g., random), or have any suitable arrangement. Antenna positions may be selected or determined from direct measurement, a design specification, global positioning information, survey information, or in any other suitable way. In some embodiments, the antenna elements 103 making up imaging array 101 are simple monopoles which, when submerged in a lossy liquid 106, provide an omni-directional radiation pattern for full target coverage and good, broadband, impedance matching. The receiving monopoles produce negligible field disturbance so that almost all scattering effects emanate from dielectric bodies. The monopoles may each have the same orientation such that only one component of the electric field is measured. For example, elements 103 may be monopoles oriented in the z direction (as shown in FIG. 1) such that only the z component of the electric field is measured. Though, other types of antenna or antenna orientations may be used.

MUT 104 is immersed in lossy liquid 106 which is confined, with imaging array 101, by a tank 102. Lossy liquid 106 dampens reflections such that the direct path between a transmitter and receiver makes the dominant contribution to the measured signal and most of the indirect paths are sufficiently attenuated that they may be ignored during processing of the microwave measurements. It has been found that a mixture of Glycerin and water provides a biologically acceptable medium into which the patient's breast can be safely immersed. The two liquids are completely missible and their mixture ratios can be easily adjusted to match breast tissue properties as close as possible without prior information. A ratio of 80:20 glycerin to water has been found suitable for a dense breast but a 86:14 ratio for fattier breasts. In some embodiments, a different lossy liquid is used or a lossy liquid may not be used at all.

The hardware control system 107 controls each of the antenna elements 103 in imaging array 101. Hardware control system 107 is configured to provide an excitation signal on one antenna element and measure the received signal on each of the remaining elements. The excitation signal may be sinusoidal of a frequency between about 500 MHz and 3 GHz, between 50 MHz and 20 GHz, of any other suitable frequency or within any other suitable frequency range. In some embodiments, a multi-frequency signal may be used. Though, any suitable excitation signal may be used. Multiple excitation signals may be sequentially provided to the excited antenna element and measurements on the receiving elements may be taken for each excitation signal. In one embodiment, data are taken at 11 different frequencies. Though, any suitable number of frequencies or more generally, excitation signals, may be used.

Exciting one element and measuring the received field on each of the other elements is repeated with each element, at least once, designated as the transmitting antenna. For an N antenna imaging array, for example, N(N-1) measurements may be collected. (By reciprocity, half of these measurements are unique.) The presence of lossy liquid 106 ensures that signals collected by the receiving elements are dominated by waves propagating through the imaging region, and that all other reflections and various multipath signals remain negligible. Imaging array 101 may be calibrated by taking measurements when MUT 104 is not present. For example, “homogeneous field” measurements are taken with just lossy liquid 106 present in tank 102 (and without MUT 104) may be used for calibration of imaging array 101.

A secondary effect of these losses is to significantly weaken the received signals, requiring reception channels to accurately capture very low power signals. For example, in an imaging system built by Microwave Imaging System Technologies Inc. (MIST), Hanover, Mass., the hardware control system was able to reliably measure received signals attenuated down to 140 dBm. This effectively translates into higher operating frequencies and, therefore, improved resolution. Such a low noise floor also requires significant channel-to-channel isolation. In the MIST system referenced above, channel-to-channel isolation was 150 dB. These signal performance values are exemplary and the actual values required for a particular application and configuration may vary.

Imaging system 100 may have the capability to move antenna elements 103 of imaging array 101 up and down in the z direction as labeled. Elements 103 may be moved as a group, controlled individually, or in some other configuration. For example, the MIST prototype allowed for cross-plane measurements by having the antennas divided into two interleaved sets of eight antennas controlled by separate motors for their vertical positioning. It is not critical, if or how the position of the array elements may be controlled or placed.

Data for forming a 3D image may be collected by shifting antenna elements 103 up or down as a group, subgroup, or individually. For the breast examination, data are collected in each of several anatomically coronal planes. Alternatively, the MUT itself may be moved through the array.

Optionally, imaging system 100 includes a MUT locating device 108. Device 108 is used to provide information about the location of the volume occupied by the breast. (Knowing the location of the volume also provides information about the surface of the MUT.) MUT locating device 108 may be operably connected to image processing device 110 to provide breast location data.

Once the measurement data have been collected by hardware control system 107, the data are provided to image processing device 110. Image processing device 110 rapidly processes the measurement data to form a 2D or 3D image (image data) which may be stored on a computer readable medium and/or provided to an output device 109. Though, in some embodiments, image processing device may receive “measurement” data that has been simulated using a computer. The image data may represent the permittivity, ε, of the imaged region (e.g., surface or volume), the conductivity, σ, of the imaged region, both, or any other suitable parameter. In some embodiments, image processing device 110 may convert the electrical properties of the imaged region into a material property of interest, such as material density, water content, or any other material property of interest. The operation of image processing device 110 to rapidly convert measurement data into an image is described below.

In order to rapidly process the measurement data image processing device 110 may have a processor 111 and memory 113. Processor 111 may be configured to control image processing device 110 and may be operatively connected to memory 113. Processor 111 may be any suitable processing device such as, for example and not limitation, a central processing unit (CPU), digital signal processor (DSP), Graphical Processing Unit (GPU), controller, addressable controller, general or special purpose microprocessor, microcontroller, addressable microprocessor, programmable processor, programmable controller, dedicated processor, dedicated controller, or any suitable processing device. In some embodiments, processor 111 comprises one or more processors, for example, processor 111 may have multiple cores and/or be comprised of multiple microchips.

Memory 113 may be integrated into processor 111 and/or may include “off-chip” memory that may be accessible to processor 111, for example, via a memory bus (not shown). Memory 113 may store software modules that when executed by processor 111 perform desired functions. Memory 113 may be any suitable type of non-transitory, computer—readable storage medium such as, for example and not limitation, RAM, a nanotechnology-based memory, one or more floppy disks, compact disks, optical disks, volatile and non-volatile memory devices, magnetic tapes, flash memories, hard disk drive, circuit configurations in Field Programmable Gate Arrays (FPGA), or other semiconductor devices, or other tangible, non-transient computer storage medium.

It should be appreciated that the actual computational time required for processing of the measurement data to produce image data will depend on the hardware capabilities of device 110 including the capabilities of, for example, processor 111 and memory 113.

Output device 109 may be any suitable output device for providing the resultant image in a human viewable form. For example and not limitation, output device 109 may be a display or printer. Though, any suitable output device may be used. Once provided by output device 109, a technician may visually analyze the data. For example, a medical professional may determine the presence of a tumor. In some embodiments, analysis criteria may be set and the result of the evaluation of the image analysis, rather than or in addition to the image itself, may be output by output device 109.

It should be appreciated that hardware control system 107, image processing device 110 and output device 109 may be separate devices or may share common components. If hardware control system 107 and image processing device 110 are not integrated, communication between the two may be performed over a wire, wirelessly, via a portable computer-readable storage medium, or in any other suitable way.

Having provided an overview of at least one embodiment of an imaging system, the theoretical basis for improved processing speed is described. Those of skill in the art will appreciate that the described operations may be implemented as computer-executable instructions stored on a non-transitory computer-readable storage medium such that when the instruction are executed by a computer they operate to rapidly convert measurement data into image data. Though, it should be appreciated that the described operations may be implemented in any suitable way.

The inventor has recognized and appreciated that the configuration of imaging array 101 and MUT 104 can be efficiently analyzed to permit rapid processing of measurement results from array 101 into a 2D or 3D image of the MUT. It is further appreciated that in some embodiments the configuration of the imaging array and the emersion of the MUT in a suitable lossy liquid allows for several simplifications to the electromagnetic modeling of the imaged region and device. The solution provided here may be used generally, though, if simplifying assumptions about the electromagnetic fields and/or geometry of the problem may be made computation time may be significantly reduced.

One aspect of the analysis makes use of the discrete dipole approximation (DDA) combined with the Gauss-Newton solution of the normal equation. The discrete dipole approximation was introduced in Purcell '73 as a way to predict scattering from astronomical dust particles. The Gauss-Newton solution of the normal equation is documented in Meaney '07. The minimization problem is written as the normal equation:

( J̅ J̅^{T}+λ I̅)Δk²= J̅^{T}ΔĒ (1)

where Δk² denotes the increment in k² with k²=ω²εμ₀+iωμ₀σ being the complex wavenumber squared which gathers the two unknowns ε (dielectric permittivity) and σ (electric conductivity), ω=2πf where f is the frequency in Hz, J̅ and J̅^{T} are the Jacobian matrix and its transpose, respectively, I̅ is the identity matrix, λ is a regularization parameter, and ΔĒ is the difference between the measured and computed electric fields. Here the array elements are assumed to be monopoles aligned in the z direction, such that only the z component of the electric field is measured. Those of skill in the art will appreciate that any suitable antenna and orientation may be used. The values of ε and σ are iteratively updated across the imaging region in order to minimize ΔĒ.

The Jacobian matrix, J̅, which gathers the derivative of the electric field with respect to the unknowns (permittivity and conductivity at each dipole), may be computed in view of the analytical expression of the electric field with these parameters. The exact formula is provided in Eq. (7) below.

In addition, a logarithmic version of Eq. (1) allows the amplitude and phase differences of the electric field to be minimized rather than its real and imaginary parts (see Meaney '07). In this way, the problem is linearized and phase information useful for the recovery of ε and σ is retained. A comparison between the present solution and the prior methodology (which seeks at minimizing the complex values of the electric field directly, without phase unwrapping) reveals important advantages of the logarithmic implementation: faster convergence, insensitivity to initial guess (a crucial property in imaging application where prior information is not likely to be available), robust even for increasing frequencies (thus lending itself to improved resolution), all of which contribute to producing significantly more accurate images in cases of large and/or strong scatterers (Grzegorczyk '11).

The DDA is used as the forward solver required at each iteration and representing an important portion of the overall computation burden of the algorithm. The discretization of scatterers such as biological tissues, civil engineering structures, and other test materials into dipoles is well suited for modeling heterogeneous structures, ideal for our configuration and considerably faster than a numerical approach (typical forward solutions are obtained in seconds instead of minutes). The electric field is computed from the collective sum of interacting dipoles whose elementary field at position r̅ᵢ due to a dipole at r̅ⱼ is governed by

$$\overline{E} ( \overline{{r}_{ij}} ) = \frac{1}{4 π {ε}_{b}} \frac{{e}^{i {k}_{b} {r}_{ij}}}{{r}_{ij}} [ \begin{matrix}{k}_{b}^{2} ( {\hat{r}}_{ij} \times {\overline{P}}_{j} ) \times {\hat{r}}_{ij} + \\ [ 3 {\hat{r}}_{ij} ( {\hat{r}}_{ij} \cdot {\overline{P}}_{j} ) - {\overline{P}}_{j} ] \frac{1 - i {k}_{b} {r}_{ij}}{{r}_{ij}^{2}}\end{matrix} ] \qquad ( 2 )$$

where P̅ⱼ is the polarization vector of the dipole at r̅ⱼ, k_{b}=ω\sqrt{ε_{b}μ₀} with ε_{b} being the permittivity of the background media, and \hat{r}ᵢⱼ=( r̅ⱼ− r̅ᵢ)/| r̅ⱼ− r̅ᵢ| is the unit vector between point i and point j separated by a distance rᵢⱼ=| r̅ⱼ− r̅ᵢ|. The background media may be a lossy liquid, air or any suitable media. For example, if a lossy liquid is used, ε_{b} is chosen as the complex permittivity of the lossy liquid. Note that where i appears as an subscript it is an index; otherwise i=\sqrt{−1}.

Eq. (2) can be cast as a matrix operation Ē( r̅ᵢⱼ)=Āᵢⱼ· P̅ⱼ and the collective response of all dipoles in response to the incident field is written as

$$\overline{E} ( \overline{{r}_{i}} ) = {\sum }_{j \neq i} \overline{{A}_{ij}} \cdot \overline{{P}_{j}} \qquad ( 3 )$$

to which the incident field Ē_{inc}( r̅ᵢ) needs to be added in order to obtain the total field. The unknown P̅ⱼ are computed by solving a similar matrix equation where the self-term is given by Ē( r̅ᵢ)= P̅ᵢ/αᵢ and the polarizability αᵢ of dipole at position r̅ᵢ whose effective volume, νᵢ, is related to the unknowns by

$${α}_{i} = 3 {ε}_{b} {v}_{i} \frac{{\tilde{ε}}_{i} - {ε}_{b}}{{\tilde{ε}}_{i} - {ε}_{b}} , {\tilde{ε}}_{i} = {ε}_{0} ( {ε}_{i} + i \frac{{σ}_{i}}{ω {ε}_{0}} ) \qquad ( 4 )$$

The master equation of the DDA is therefore

$${α}_{i} {\overline{E}}_{inc} ( \overline{{r}_{i}} ) = \overline{{P}_{i}} - {α}_{i} {\sum }_{j \neq i} \overline{{A}_{ij}} \overline{{P}_{j}} . \qquad ( 5 )$$

In this formulation the computation of the Jacobian matrix remains analytical. This is seen by writing the total field at receiver l in a similar way to Eq. (5). Denoting the interaction matrix by B̅ᵢⱼ and writing the total field at receiver l as, the expression is:

Ēₜₒₜ( r̅ₗ)=Ē_{inc}( r̅ₗ)+ B̅ₗᵢ· P̅ᵢ (6)

Omitting the indices for simplicity, the Jacobian matrix with respect to ξ where ξ={ε, σ} is directly given by

$$\frac{\partial {\overline{E}}_{tot}}{\partial ξ} = \overline{\overline{B}} \cdot \frac{\partial \overline{P}}{\partial ξ} = \overline{\overline{B}} \cdot {( \overline{\overline{I}} - α \overline{\overline{A}} )}^{- 1} [ \frac{\partial α}{\partial ξ} ( {\overline{E}}_{inc} + \overline{\overline{A}} \cdot \overline{P} ) ] . \qquad ( 7 )$$

The computation therefore only requires the derivative of the polarizability, α, with respect to ε and σ, which is straightforward in view of Eq. (4):

$$\begin{matrix}\frac{\partial α}{\partial ε} = {ε}_{0} [ {v ( \frac{3 {ε}_{b}}{{ε}_{s} + 2 {ε}_{b}} )}^{2} ] & ( 8 ) \\ \frac{\partial α}{\partial σ} = \frac{i}{ω} [ {v ( \frac{3 {ε}_{b}}{{ε}_{s} + 2 {ε}_{b}} )}^{2} ] & ( 9 )\end{matrix}$$

These equations can be cast in a series of matrix multiplications, where the matrices themselves can be pre-computed and reused at each iteration of the Gauss-Newton iterative approach, yielding a considerable time saving. The basic system of equation can be written as

$$\begin{matrix}[ \begin{matrix}\frac{\partial ( \overline{E} )}{\partial ε} & \frac{\partial ( \overline{E} )}{\partial σ} \\ \frac{\partial ( \overline{E} )}{\partial ε} & \frac{\partial ( \overline{E} )}{\partial σ}\end{matrix} ] [ \begin{matrix}Δ ε \\ Δ σ\end{matrix} ] = [ \begin{matrix}{ε}_{0} ( \overline{V} ) & - \frac{1}{ω} ( \overline{V} ) \\ {ε}_{0} ( \overline{V} ) & \frac{1}{ω} ( \overline{V} )\end{matrix} ] [ \begin{matrix}Δ ε \\ Δ σ\end{matrix} ] \\ = [ \begin{matrix} ( Δ \overline{E} ) \\ ( Δ \overline{E} )\end{matrix} ]\end{matrix} \qquad ( 10 )$$

where [symbol] (·) and ℑ(·) denote the real and imaginary part operators, respectively, and

$$\overline{V} = {v ( \frac{3 {ε}_{b}}{{ε}_{s} + 2 {ε}_{b}} )}^{2} \overline{\overline{B}} \cdot {( \overline{\overline{I}} - α \overline{\overline{A}} )}^{- 1} [ \frac{\partial α}{\partial ξ} ( {\overline{E}}_{inc} + \overline{\overline{A}} \cdot \overline{P} ) ] . \qquad ( 11 )$$

Eq. (10) can be directly transformed into the logarithmic version of the algorithm following the procedure outlined in Meaney '01.

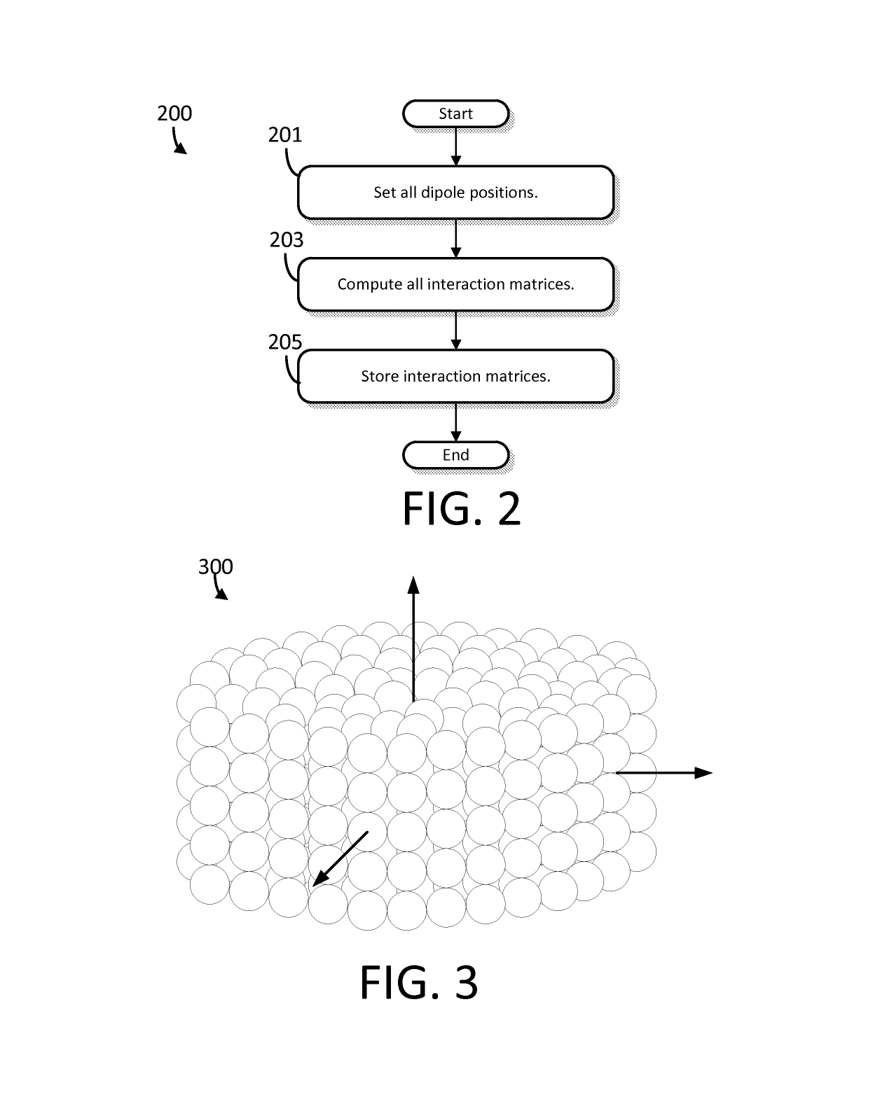

In order to expedite conversion of the measurement data into the sought image, certain computations may be performed prior to collection of measurement data. The result of the computation may be stored and used for each imaging procedure. This “offline” procedure is described with reference to method 200, shown in FIG. 2. Method 200 may be repeated for multiple variants of the imaging problem. For example, an iteration of method 200 may be performed for each of multiple excitation frequencies, different antenna positions or orientations, or for changes in any other parameter that defines the general problem to be solved. Method 200 may be implemented on a computing device such as image processing device 110 shown in FIG. 1. Though, method 200 may be implemented in any suitable way.

At step 201, the positions, r̅ᵢ, of the diploes in the imaging region are defined. This is shown conceptually by the arrangement of dipoles 300 in FIG. 3. The dipoles in general may be positioned at arbitrary positions in the coordinate space. The density of the dipoles should be sufficient to ensure accuracy of the computed electric fields and, if used, magnetic fields. The dipole positions may be irregular because of random placement, or may define any shape, regular or otherwise. In some embodiments, the dipoles are placed on a rectangular or square grid. For example, the dipoles may be placed on a square grid with uniform spacing in all three coordinate directions. If a regular grid such as a cubic lattice or rectangular grid is chosen to position the dipoles, the fast Fourier transform (FFT) may be utilized in later steps. Though, any suitable arrangement of dipoles may be used.

The number and arrangement of dipoles may also differ for 2D and 3D imaging. In an application where 2D planes are to be imaged, the number of layers of dipoles may be significantly limited as scatters from dipoles further away from the plane of interest may not make a significant contribution to the received signals (this property is related to attenuated caused by the lossy bath described above). In contrast, for 3D imaging the entire volume to be imaged may have dipoles. Typically, the space filled with dipoles will be of significant greater height in 3D imaging than in 2D imaging. The distance to the furthest dipoles above and below the imaging plane defined by the array may be determined by the attenuation of received signals refracted from this distance (e.g., inverting for those dipoles that are within a certain pre-defined fraction of the incident field power). For example, there may be no need to estimate material properties for dipoles at positions where the received signal is, for example, 20, 40, 50 or 100 dB down from the direct path signal.

In an embodiment for 3D breast imaging, about 4,500 dipole locations organized in a cylindrical fashion spanning 5 cm in height with a radius of 7 cm, i.e. located just within the radiating antennas, was found to produce good results. A sufficient number of dipoles should be selected to ensure that all field variations are properly captured with the accuracy required for the application.

At step 203, interaction matrices are computed. The interaction matrices may be independent of the polarization vector, P̅ᵢ, and may be completely defined by known quantities such as the excitation frequency, dipole positions (defined at step 200), array element positions, and background permittivity.

A first interaction matrix between all the dipoles may be computed. The first interaction matrix may later be used to compute the scattered field at the receivers.

A second interaction matrix between the transmitters and all the dipoles may also be computed. The second interaction matrix may later be used to compute the incident field at all the dipoles. If the transmitter is modeled as a simple source (e.g., a point source, line source, plane wave) the second interaction matrix may be found directly from the theoretical model of these sources. For example, in case of a scatterer in the far field, a plane wave assumption may be sufficient (i.e., an exponential at the location of interest). In some embodiments, the transmitters are modeled by one or more radiating dipoles. The antenna elements of the MIST system, for example, are each modeled by a single dipole. Though, the transmitter may be modeled in any suitable way.

A third interaction matrix between the transmitter and all the receivers may be computed. The third interaction matrix my later be used to compute the incident field at the receivers. The transmitter may be modeled similarly to that used for calculation of the second interaction matrix, above. Though any suitable model may be used.

In some embodiments, the interaction matrices may be computed for different values of fixed parameters. For example, the interaction matrices may be computed for different excitation frequencies (e.g., for use in cases where measurements are taken at multiple frequencies) or for different values of the background permittivity (e.g., for use with different lossy liquids or for different densities of dipoles in order to obtain more or less sharp images).

At step 205, the precomputed interaction matrices are stored, for example, on a computer-readable storage medium. Because the precomputed matrices are generic and not specific to a MUT, they can be later used for any MUT. In the example of breast imaging, the same precomputed interaction matrices may be used for each patient. Moreover, the same interaction matrices can be used for other applications.

Method 200 may be repeated for different configurations. For example, the dipole positions may be changed. In this way a library of precomputed interaction matrices may be formed. If the breast location and boundary is known, for example from MUT locating device 108 (FIG. 1), the polarization of the dipoles outside the breast volume may be of known value. The polarization may be defined as being proportional to ε-ε_{b} such that the polarization of the outside dipoles is 0 as here ε=ε_{b} and thus make no contribution to the scattered field and therefore do not contribute to dipole-to-dipole interactions in the interaction matrices. (Note that here ε and ε_{b} are generally complex numbers representing both permittivity and conductivity.) As a result, the dipoles outside the breast region, by definition of the polarization and of ε_{b}, may be eliminated. Thus, while the interaction matrices will need to be recomputed, the number of dipoles in the computation may be reduced potentially reducing the overall computation time. On the other hand, if an accelerated method is used such as FFT, all the dipoles on the rectangular grid are necessary, even if they are outside the breast boundary. But the polarization of these dipoles can be enforced a posteriori.

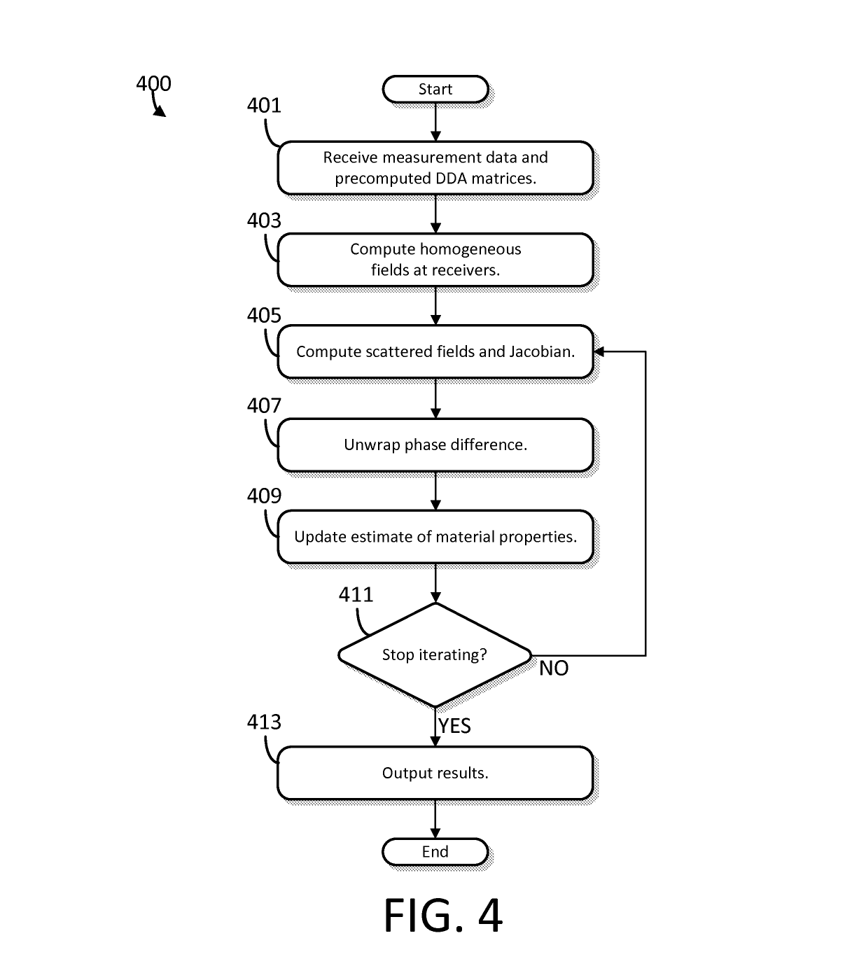

Regardless of whether the interaction matrices are precomputed generically, or reflect specific knowledge about the location of the breast, method 400, shown in FIG. 4, is performed after sensor data collection from the imaging array by the hardware control system. Method 400 may be implemented on image processing device 110 shown in FIG. 1. Though, method 400 may be implemented on any suitable computing device or in any suitable way. Steps of method 400 that are independent may be performed at any suitable time. For example, independent steps may be performed sequentially (e.g., as shown), in parallel, or in any suitable way. For 3D breast imaging data taken with a 16 element imaging array at 7 z positions an 11 frequencies and reconstructed using about 4,500 dipoles, imaging time was under 20 minutes on a 2.8 GHz Intel Xeon processor. Though, imaging times will vary depending on the application and capabilities of the image processing device.

At step 401, the measurement data along with the precomputed interaction matrices is received by the image processing device. The matrices may be preselected from a library formed by method 200. In some embodiments, the measurement data are received directly from the hardware control system for the imaging array or are read from a storage medium. Though, the measurement data may be received in any suitable way.

The measurement data may be provided for a single set of array element positions (e.g., for on “z” position of the imaging array) or for a complete scan (e.g., with measurement data collected for multiple “z” positions). In the latter case a full 3D image may be formed directly. In the former case a single 2D image may be formed; method 400 may then be repeated (e.g., for each “z” position) and a 3D image may be formed by the combining each of the 2D results. The information contained in the measurement data may represent the field at the receiver as an amplitude and phase, a real and imaginary component, or in any suitable way.

The precomputed interaction matrices may be received by loading the information from a storage medium or in any other suitable way. If precomputed interaction matrices are not being used, for example, because they are not available or because specific information about the breast is available from a MUT locating device (e.g., see MUT locating device 108, FIG. 1) to select dipole locations, method 200 described with reference to FIG. 2 may be performed to generate the interaction matrices.

At step 403, the homogeneous fields at the receivers are computed. The homogeneous fields represent the response when the entire imaging region is defined by the background material with properties ε_{b}. The homogeneous fields may be computed for every antenna excitation and every antenna position used during collection of the measurement data. This may include, for example, each of multiple frequencies of excitation for each transmitter/receiver combination.

If the dipole positions have a regular grid such as a cubic lattice or rectangular grid, the FFT and Block-Toeplitz form of the matrix may be exploited to further expedite computation. Specifically, Eq. (2) may be written in a matrix form as Āᵢⱼ· P̅ⱼ where matrix Ā depends on rᵢ and rⱼ through their difference so that Āᵢⱼ depends only on the index difference: Āᵢⱼ=Āᵢ₋ⱼ if i≠j or Āᵢⱼ=0 if i=j. With the cubic lattice, the exponential term takes the form exp(iknd) where exp(·) is the exponential function, k is the wavenumber defined above, n is an integer, and d is the lattice constant. The matrix equation therefore becomes a convolution which can be evaluated using an FFT. For a cubic lattice, the complexity is reduced to O(N log N) instead of O(N²).

Steps 405, 407, and 409 are iterated steps. They may be repeated in accordance with a decision made at step 411.

At step 405, the fields at the receivers and the dipoles are computed using the discrete dipole approximation (DDA). During the first iteration an initial guess for the permittivity and conductivity of all the unknown dipoles is made. In some embodiments, the initial guess is to assume all the dipoles have the properties of the background, ε_{b}. Once the initial guess is made, the fields at each of receivers are computed. Specifically, three matrices may be computed: one for the scattered field, one for the incident field at the dipole locations, and one for the incident field at the receiver locations. These results may be used to provide the total field at the each dipole location and at each receiver location. Computation of the fields is performed for each transmitter/receiver combination and for each excitation frequency.

The Jacobian matrix, J̅ is completely determined and may also be computed at step 405. The Jacobian matrix may be computed analytically, numerically, semi-analytically, or any suitable way. The adjoint method or any other suitable method for evaluating the derivatives of Eq. (7) may be used. The polarizabilities determined by the initial guess and subsequent updates in the iterative process may be used to evaluate the Jacobian matrix. In embodiments where the dipoles have a regular grid such as a cubic lattice or a rectangular grid the fast Fourier transform (FFT) may be used to further simplify and expedite computation. Similarly, the Block-Toeplitz form of the matrix may be exploited (Barrowes '01).

During second and subsequent iterations at step 405 the scattered field at the receivers and dipoles are computed for the updated material properties. (The updated material properties for the next iteration are determined as part of step 409.) The Jacobian matrix is computed for the new guess of the material properties.

At step 407, the phase difference at each receiver is computed as the difference of the phase calculated at step 405 and the phase of the homogeneous field calculated at step 403. The phase difference is the difference in phase between the current estimate of the electric field and that of the homogenous electric field computed at step 403 (<Ē_{current}−<Ē_{homogenous}). At the first iteration, the phases are equal (<Ē_{current}=<Ē_{homogenous}) so the phase difference starts at zero. The incremental updates of the material properties are designed to be small enough to ensure that no jumps of 2π are missed. Thus in an iteration where the phase difference is smaller than that of the previous iteration, the increment can be assumed to be less than 2π. For example, if the phase at a given iteration is 350 degrees and at the next iteration is 20 degrees, we adjust the total phase shift to be 370 degrees. If in the next increment the phase shift was 25 degrees, the total phase shift would become 395 degrees. In some embodiments the unwrapped phase is estimated by frequency hopping. (reconstructing the images are lower frequencies first where large wavelengths ensure phase continuity, then utilizing low frequency images as initial guesses at high frequency).

At step 409, the estimate of the material properties is updated using a suitable inversion algorithm. The inversion algorithm takes as inputs the measurement data received at step 401, the complex field at the receivers computed at step 405 for the present iteration, the complex homogeneous field at the receivers computed at step 403, the unwrapped phase computed at step 407, and the Jacobian matrix. Both the measured and the computed fields may be calibrated using the respective homogeneous field data.

From this information ΔĒ, from Eq. (1), may be calculated as the difference between the measured field minus the computed field. In some embodiments, the logarithmic version Eq. (1) is used and accordingly ΔĒ is taken in a log/phase format as is the Jacobian and its transform. In the example of the logarithmic version, the amplitude of the change in field (i.e., |ΔĒ|, may be evaluated as:

[log(|E_{measured with target}|)−log(|E_{homogeneous measured}|)]−[log(|E_{computed with target}|)−log(|E_{homogeneous computed}|)] (11)

where log(·) is the natural logarithm. Similarly, the phase of the change in field (i.e., <ΔĒ) may be evaluated as:

[<E_{measured with target}−<E_{homogeneous measured}]−[<E_{computed with target}−<E_{homogeneous computed}] (11)

The regularization parameter, λ, may be determined in ways known in the art such as those described in Meaney '07. The only remaining term from Eq. (1) is Δk² which may be found by matrix inversion of Eq. (1). The term Δk² represents the increment in Δk² between the present iteration the next iteration. As Δk² is directly proportional to epsilon and sigma (recall k²=ω²εμ₀+iωμ₀σ) the matrix inversion gives the increment in epsilon and sigma directly.

The inversion algorithm may use the Gauss-Newton technique. The increment size for the material properties used by the Gauss-Newton technique may be controlled. In some embodiments, the maximum increment is set to a lower value during the early iterations and is increased in later iterations. The maximum increment may be ramped up from iteration to iteration. The increment should be small enough, however, to avoid having a phase variation of greater than 2π as this will cause the phase to be unwrapped improperly and lead to erroneous results. Control of the increment size may be done by limiting the increment in the updates of the unknown material properties, by using a sweeping frequency technique (described below), or by both. Though, any suitable method may be used. Once the normal equations are built, a matrix inversion which gives the increment in the material properties (ε and σ) from iteration to iteration is performed.

In some embodiments, a sweeping frequency technique may be used to track the evolution of phase. The sweeping frequency technique tracks the evolution of phase by looking at the field at a given receiver at a low frequency where the phase changes slowly and the possibility of a phase jump is low. The frequency is the slowly ramped up while the fields are tracked for jumps. This may be repeated for each receiver.

In some embodiments, the Gauss-Newton technique may be regularized by a Tikhonov method as well as Levenberg-Marquardt as described in Meaney '07. Specifically, a two step regularization may be performed before solving the system which also involves a matrix normalization. More explicitly, after the system is built (assuming H̅= J̅ J̅^{T} or H̅= J̅^{T} J̅):

(1) Perform Tikhonov regularization on H̅ as shown in Eq. (1), H̅+λ I̅. The regularization parameter, λ, is computed at each iteration and is directly proportional to J̅^{T}ΔĒ, the right hand side of Eq. (1), i.e. the mismatch between the measured and computed quantities. Thus, at early iterations the regularization is stronger and at later iterations, where the mismatch is reduced and the estimated dipole values of epsilon and sigma improve, the ill-conditioning of the system is reduced and less regularization is required. As shown, Tikhonov regularization may be used to perform the regularization. Though, regularization may be performed in any suitable way.

(2) Normalize the new matrix so that the diagonal terms are 1.

(3) Perform a Levenberg-Marquardt regularization: further weigh the diagonal in order to achieve a good conditioning number of the new matrix. In some embodiment, this second regularization is not performed (the weight is set to zero).

(4) Solve the system and readjust the result by the normalization factors above.

At step 411, a determination is made whether to stop iterating through steps 405, 407 and 409. If it is determined to perform another iteration, method 400 returns to step 405. If it is determined to stop iterating, method 400 continues to step 413. In some embodiments, the determination is based on a fixed number of iterations. For example, after 10 (or 20) iterations method 400 continues to step 413. Though, iteration may stop after any suitable number of iterations. In some embodiments, the received fields are computed and compared to the measured fields. If the differences are lower than a certain error threshold, method 400 continues to 413, otherwise it returns to step 405. In yet some other embodiments a combination of criteria are used. For example, the determination may be made based on satisfaction of an error threshold but no fewer than a predetermined number of iterations. Though, the determination at step 411 may be made in any suitable way.

At step 413, the estimated material properties are provided to an output device such as output device 109 discussed in connection with FIG. 1. The estimated material properties may be the estimated permittivity or conductivity values for the imaging region. Though, in some embodiments, the estimated permittivity and conductivity values may be used to estimate density, or another property of direct interest.

In some embodiments, at step 413 the material properties are presented on a display or printed onto a paper or other medium. The image may be stored onto a computer-readable storage medium. The material properties may be represented as a 3D image, a 2D image, a table, or in any suitable way. As discussed above, a 3D image may be formed directly by estimating material properties for the complete imaging area. The display may show information to the operator in the form of an image, as numerical values of properties at locations chosen by the operator (using a mouse or other device), as statistical information on properties over user-defined regions of interest, or in any other suitable way or combination thereof. The image may be shown as a 3D rendering, rotations, iso-surfaces, slices, angles of view, opacities, or in any other suitable representation.

Alternatively, a 3D image may be formed by stitching together multiple 2D images. The dipoles for each 2D imaging may overlap and the 3D image may be formed by performing a weighted averaging as function of the distance to the “zero” antenna plane. In other words, the dipoles in the plane of the antenna are likely to have more accurate estimates of the material properties than those dipoles further away from the antenna plane. But those dipole s away from the zero plane be in the plane of the antennas for an adjacent 2D reconstruction (i.e., where the antennas are moved up or down). The weighted average may favor the material property of the dipole which is closer to its respective zero plane.

On the other hand, a 2D image may be output by performing a 2D inversion directly at step 409, by direct extraction from the 3D image, or obtained by directional averaging (i.e. an averaging of all the permittivity or conductivity values along a certain direction). In imaging a breast for example, it is common to present information in the coronal or sagittal direction. Though, any suitable direction may be selected. In some embodiments the estimated material properties are stored on a computer-readable storage medium.

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Accordingly, the foregoing description and drawings are by way of example only.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

In this respect, it should be appreciated that one implementation of the above-described embodiments comprises at least one computer-readable medium encoded with a computer program (e.g., a plurality of instructions), which, when executed on a processor, performs some or all of the above-discussed functions of these embodiments. As used herein, the term “computer-readable medium” encompasses only a computer-readable medium that can be considered to be a machine or a manufacture (i.e., article of manufacture). A computer-readable medium may be, for example, a tangible medium on which computer-readable information may be encoded or stored, a storage medium on which computer-readable information may be encoded or stored, and/or a non-transitory medium on which computer-readable information may be encoded or stored. Other non-exhaustive examples of computer-readable media include a computer memory (e.g., a ROM, a RAM, a flash memory, or other type of computer memory), a magnetic disc or tape, an optical disc, and/or other types of computer-readable media that can be considered to be a machine or a manufacture.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The inventor expresses his appreciation to Microwave Imaging System Technologies Inc. for providing measurement data used for validation.

The following citations are provided:

Meaney '288—P. M. Meaney and K. D. Paulsen, Two-dimensional microwave imaging apparatus and methods, U.S. Pat. No. 5,841,288, Nov. 4, 1998.

Purcell '73—E. M. Purcell and C. R. Pennypacker, Scattering and absorption of light by nonspherical dielectric grains, The Astrophysical Journal, 186: 705-714, Dec. 1, 1973.

Meaney '07—P. M. Meaney, Q. Fang, T. Rubaek, E. Demidenko, K. D. Paulsen, Log transformation benefits parameter estimation in microwave tomographic imaging, Med. Phys. 34, 2014 (2007).

Grzegorczyk '11—T. M. Grzegorczyk, P. M. Meaney, S. I. Jeon, S. D. Geimer, K. Paulsen Importance of phase unwrapping for the reconstruction of microwave tomographic images, Biomedical Optics Express, vol. 2, no. 2, pp. 315-330, Feb. 1, 2011.

Meaney '01—P. M. Meaney, K. D. Paulsen, B. W. Pogue, M. I. Miga, IEEE Trans. Med. Imaging 20, 104 (2001).

Barrowes '01—B. E. Barrowes, F. L. Teixeira, J. A. Kong, Fast algorithm for matrix-vector multiply of asymmetic multilevel block-Toeplitz matrices in 3-D scattering, Microwave and Optical Technology Letters, vol. 31, no 1, Oct. 5, 2001.

What is claimed is:

1) A non-transitory, computer-readable storage medium having computer-executable instructions that, when executed perform a method of imaging a test material from microwave measurement data, the method comprising acts of:
(a) receiving the microwave measurement data;
(b) computing a field at each of a plurality of dipole locations and at each of a plurality of receiver locations for a guess of material properties;
(c) computing a Jacobian matrix using the computed field and the guess of the material properties;
(d) estimating a total phase difference at each of the plurality of receiver locations from the computed scattered field and a phase calculated for a homogeneous medium;
(e) determining a new guess of the material properties based on the total phase difference, the Jacobian matrix and the received measurement data;
(f) repeating steps (b) through (e) until an end criteria is met; and
(g) outputting a representation of the material properties.

2) The computer-readable storage medium of claim 1, wherein the measurement data comprises measured electromagnetic fields at each of the plurality of receiver locations.

3) The computer-readable storage medium of claim 1, wherein act (c) comprises computing the Jacobian matrix analytically.

4) The computer-readable storage medium of claim 1, wherein act (b) comprises:
loading one or more precomputed interaction matrices; and
computing the scattered field from the guess of the material properties and the one or more precomputed interaction matrices.

5) The computer-readable storage medium of claim 4, wherein the one or more precomputed interaction matrices comprise:
(1) interaction matrix between all dipoles in order to compute the scattered field,
(2) interaction matrix between the transmitters and receivers in order to compute the incident field at each receiver, and
(3) interaction matrix between the transmitters and all the dipoles, in order to compute the incident field from a transmitter at a given dipole location.

6) The computer-readable storage medium of claim 1, wherein act (b) comprises positioning each of the plurality of dipoles on a regular grid and using a fast Fourier transform to expedite matrix multiplication.

7) The computer-readable storage medium of claim 1, wherein act (b) comprises positioning each of the plurality of dipoles on a regular grid and simplifying execution by exploitation of a matrix Block-Toeplitz format.

8) The computer-readable storage medium of claim 1, wherein the test material comprises a human, female breast.

9) The computer-readable storage medium of claim 1, wherein the test material comprises a civil engineering structure.

10) The computer-readable storage medium of claim 1, wherein the method of imaging the test material further comprises:
receiving position information for the test material volume;
identifying dipoles positioned outside the test material volume; and
fixing the material properties of each of the dipoles identified as outside the test material volume to the material properties of a background material.

11) A non-transitory, computer-readable storage medium having computer-executable instructions that, when executed perform a method of imaging material properties of a test material from microwave measurement data, the method comprising acts of:
(a) receiving the microwave measurement data;
(b) translating the microwave measurement data into material properties of the test material by:
(i) estimating fields for a guess of the material properties using a discrete dipole approximation (DDA),
(ii) updating the guess by comparing the estimated fields with the microwave measurement data,
(iii) iterating (i) and (ii) until an end condition is met; and
(c) outputting a representation of the material properties.

12) The computer-readable storage medium of claim 11, wherein the guess is updated at (b)(ii) using a non-linear solver.

13) The computer-readable storage medium of claim 11, wherein the guess is updated at (b)(ii) using a Gauss-Newton method and a Jacobian matrix is evaluated analytically for the Gauss Newton method.

14) The computer-readable storage medium of claim 11, wherein outputting a representation of the material properties comprises providing an image of the material properties in a human viewable format.

15) The computer-readable medium of claim 11, wherein translating the microwave measurement data into material properties comprises:
separately imaging each of a plurality of image planes using separately computed dipoles; and
forming a 3D representation of the image region by combing each of the separately imaged planes using a weighted average of the dipole material properties computed for each separate image.

16) The computer-readable storage medium of claim 15, wherein the plurality of dipoles for each separately imaged plane cover a region smaller than the overall 3D imaging region.

17) The computer-readable storage medium of claim 11, wherein translating the microwave measurement data into material properties comprises generating a 3D representation of the material properties.

18) The computer-readable storage medium of claim 11, wherein translating the microwave measurement data into material properties further comprises:
forming a 2D image by directionally averaging in a direction of interest; and
providing an image of the material properties in a human viewable format.

19) The computer-readable storage medium of claim 18, wherein the direction of interest is one of a sagittal or axial directions.

20) A method of constructing a plurality of interaction matrices, the method comprising:
operating a processor to:
define a plurality of dipole locations;
compute a plurality of interaction matrices for the dipole locations; and
store the plurality of interaction matrices on a non-transitory computer readable storage medium.

21) The method of claim 20, wherein operating the processor to compute the plurality of interaction matrices comprises:
computing a first interaction matrix between all dipoles in order to compute the scattered field,
computing a second interaction matrix between the transmitters and receivers in order to compute the incident field at each receiver, and
computing a second interaction matrix between the transmitters and all the dipoles, in order to compute the incident field from a transmitter at a given dipole location.

22) The method of claim 20, wherein the plurality of interaction matrices are computed for a plurality of different excitation frequencies, a plurality of different dipole densities, and a plurality of different antenna configurations.

23) The method of claim 20, wherein the dipole positions are on a regular grid.

24) The method of claim 20, wherein the dipole positions are irregular.

25) A method of operating a computer, the method comprising:
operating a processor to:
(a) receive microwave measurement data;
(b) translate the microwave measurement data into material properties of a test material by:
(i) estimating fields for a guess of the material properties using a discrete dipole approximation (DDA),
(ii) updating the guess by comparing the estimated fields with the microwave measurement data,
(iii) iterating (i) and (ii) until an end condition is met; and
(c) outputting a representation of the material properties.

26) An image processing device comprising:
a processor configured to perform acts of:
(a) receiving the microwave measurement data;
(b) translating the microwave measurement data into material properties of the test material by:
(i) estimating fields for a guess of the material properties using a discrete dipole approximation (DDA),
(ii) updating the guess by comparing the estimated fields with the microwave measurement data,
(iii) iterating (i) and (ii) until an end condition is met; and
(c) outputting a representation of the material properties.
